(19) United States
(12) Patent Application Publication
(10) Pub. No.: US 2006/0018853 A1
(43) Pub. Date: Jan. 26, 2006

(54) Cosmetic pack preparation

(75) Inventor: Soichiro Watanabe, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kose Corporation, Tokyo (JP)

(21) Appl. No.: 10/534,976

(22) PCT Filed: Oct. 6, 2003
(86) PCT No.: PCT/JP03/12773

§ 371 Date: May 16, 2005

(30) Foreign Application Priority Data
Nov. 18, 2002 (JP) 2002-333010

(51) Int. Cl. A61K 8/81; A61K 8/29

(52) U.S. Cl. 424/63; 424/70.11

(57) ABSTRACT

A cosmetic pack preparation comprising (a) 5-20 wt % of polyvinyl alcohol and (b) 0.01-10 wt % of atitanium-titanium dioxide sinter is disclosed. The cosmetic pack preparation enables distinct identification of areas which the cosmetic composition was applied to or was peeled from and the skin cleaning effect, imparts a favorable sensation upon use such as ease of application and peeling, and excels in preservation stability with passage of time.

### TECHNICAL FIELD

The present invention relates to a film forming-type cosmetic pack preparation which enables distinct identification of areas which the cosmetic composition was applied to or was peeled from and the skin cleaning effect, imparts a favorable sensation upon use such as ease of application and peeling, and excels in preservation stability with passage of time.

### BACKGROUND ART

Conventionally, cosmetic pack preparations have been widely used to cover the skin temporarily for a certain period of time in order to supply moisture to the skin and suppress vaporization of subcutaneous moisture and to make skin flexible. Various cosmetic pack preparations are commercially available. As the form of the pack agent, there are a peel-off type in which a film formed by coating and drying is peeled off, a type in which a creamy or muddy agent is applied to the skin and wiped off or washed away, a type using a nonwoven fabric impregnated with a lotion, and the like. Among these, the peel-off type pack has an effect of removing soils adhered to the skin due to the adsorption effect of the film-forming agent and eliminating old horny layers. Polyvinyl alcohol can be given as a typical film-forming agent. Japanese Patent Application Laid-open No. 10-245320, for example, proposed the addition of fine particles to a peel-off type pack using a polyvinyl alcohol as a film-forming agent to increase the drying speed or improve the film strength.

A color pigment such as titanium oxide, red iron oxide, yellow iron oxide, or black iron oxide is added to the cosmetic pack containing polyvinyl alcohol as a film-forming agent to ensure clear identification of the applied areas and peeled areas and the skin cleaning effect.

However, in many cases, a cosmetic pack in which such a color pigment is added has impaired dispersion stability due to surface activity of the color pigment, resulting in impaired preservation stability of the cosmetic preparation.

As a means to solve such a problem associated with the use of a color pigment, it is possible to use a water-soluble dye as a coloring agent. The use of a water-soluble dye in an amount to secure a sufficient covering effect, however, produces another problem of staining the skin.

Moreover, the addition of other active components such as a water-soluble ascorbic acid derivative or a water-soluble glycyrrhizic acid derivative to provide a cosmetic pack preparation with a whitening effect or an anti-inflammatory effect reduces dispersibility of a color pigment and impairs preservation stability of other effective components.

Therefore, development of a film-forming type cosmetic pack preparation enabling clear identification of the applied areas or peeled areas and the skin cleaning effect, imparting a favorable sensation upon use such as ease of application and peeling, and excelling in preservation stability with passage of time has been desired.

### DISCLOSURE OF THE INVENTION

In view of this situation, the inventor of the present invention conducted extensive studies. As a result, the inventor has found that a film-forming type cosmetic pack preparation satisfying all of the above-mentioned requirements can be obtained by using a titanium-titanium dioxide sinter as a color pigment and combined this material with other components. This finding has led to the completion of the present invention.

Specifically, the present invention provides a cosmetic pack preparation comprising the following components (a) and (b):

(a) 5-20 wt % of polyvinyl alcohol and

(b) 0.01-10 wt % of a titanium-titanium dioxide sinter.

The present invention also provides a cosmetic pack preparation comprising, in addition to the above components (a) and (b), an anionic surfactant, particularly, at least one anionic surfactant selected from the group consisting of a phosphate-based anionic surfactant, a sulfonate-based anionic surfactant, and a sulfate-based anionic surfactant as a component (c).

The present invention further provides a cosmetic pack preparation comprising, in addition to the components (a) and (b), a water-soluble thickener, particularly, a swelling clay mineral and/or amorphous fine particles of silicic anhydride, as a component (d).

The present invention still further provides a cosmetic pack preparation comprising, as the polyvinyl alcohol of the above component (a), a mixture of (A) a polyvinyl alcohol of which the aqueous solution with a concentration of 4 wt % has a viscosity at 20° C. of 1 mPa.s or more, but less than 30 mPa.s and (B) a polyvinyl alcohol of which the aqueous solution with a concentration of 4 wt % has a viscosity at 20° C. of 30 mPa.s or more, but less than 70 mPa.s.

### BEST MODE FOR CARRYING OUT THE INVENTION

The constitution of the present invention will be described.

The polyvinyl alcohols used as the component (a) in the present invention are known in the art as cosmetic components and are abundantly used in peel-off type cosmetic pack preparations. Many types of polyvinyl alcohols classified according to the degrees of polymerization and saponification are commercially available. An appropriate class of polyvinyl alcohol can be used according to application and characteristics of the pack. The polymerization degree of polyvinyl alcohol is usually indicated by the viscosity of an aqueous solution with a concentration of 4 wt % measured at 20° C.

Polyvinyl alcohol with any degree of viscosity can be used in the present invention. To ensure a favorable sensation during use such as ease of application and peeling, a mixture of (A) a polyvinyl alcohol of which the aqueous solution with a concentration of 4 wt % has a viscosity at 20° C. of 1 mPa.s or more, but less than 30 mPa.s and (B) a polyvinyl alcohol of which the aqueous solution with a concentration of 4 wt % has a viscosity at 20° C. of 30 mPa.s or more, but less than 70 mPa.s is preferably used.

As examples of the polyvinyl alcohol (A) with a viscosity of 1 mPa.s or more, but less than 30 mPa.s, PVA-103, 110, 205, 210, 217, 613, 706 (manufactured by Kuraray Co., Ltd.), and the like can be given. Of these, polyvinyl alcohols with a viscosity of 3-10 mPa.s are particularly preferable.

As examples of the polyvinyl alcohol (B) with a viscosity of 30 mPa.s or more, but less than 70 mPa.s, PVA-120, 124, 220, 224, 228, (manufactured by Kuraray Co., Ltd.), and the like can be given. Of these, polyvinyl alcohols with a viscosity of 40-60 mPa.s are particularly preferable.

The saponification degree of the component (a) is a value dependent on the proportion of saponification of the acetyl group in polyvinyl acetate at the time of manufacturing and is broadly divided into a complete saponification-type in which the acetyl groups are saponified almost completely and a partial saponification-type in which some amount of acetyl groups are left unsaponified. Although either type can be used in the present invention, the partial saponification-type is more preferable.

The amount of the component (a) used in the cosmetic pack preparation of the present invention is 5-20 wt % (hereinafter referred to simply as “%”), and particularly 7-15%. If the content of polyvinyl alcohol is less than 5 wt %, the resulting cosmetic pack preparation tends to drip during application, film formability is insufficient, and the film can be peeled off only with difficulty. If the content exceeds 20%, the cosmetic pack preparation can be spread only with difficulty due to the high viscosity and exhibits impaired preservation stability due to gelatinization with passage of time. Although there are no specific limitations to the weight ratio of the polyvinyl alcohol (A) and the polyvinyl alcohol (B) in the component (a), a ratio in the range of 0.05-2 is preferable to ensure excellent sensation during use, with a range of 0.1-1.5 being more preferable.

The component (a) is used dissolved in water. The amount of water to dissolve the component (a) is preferably 70-90% of the total amount of the cosmetic pack preparation.

The titanium-titanium dioxide sinter used as the component (b) in the present invention is added to increase the covering effect of the cosmetic pack preparation, to make it easy to identify the applied areas and peeled areas and the skin cleaning effect, and to increase the drying speed and film strength, thereby rendering the film easy to be peeled off.

Since the particles of titanium-titanium dioxide sinter used as the component (b) have a particle surface activity lower than that of conventionally used titanium oxide and black iron oxide, the titanium-titanium dioxide sinter can ensure excellent dispersion stability without impairing the preservation stability of the cosmetic pack preparation containing polyvinyl alcohol. Any titanium-titanium dioxide sinter commonly used in cosmetic compositions can be used as the component (b) without any specific limitations. Titanium-titanium dioxide sinter produced by mixing fine particles of titanium and titanium oxide at a ratio of 1:5 and sintering the mixture at a temperature of 900-1000° C. under reduced pressure for 3-5 hours can be given as an example.

To ensure a good covering effect and dispersion stability of pigments, the average particle diameter of the component (b) is preferably in the range of 0.6-2.0 μm, and more preferably 0.6-1.5 μm. Particles of titanium-titanium dioxide sinter of which the surface has been treated using a commonly known method may be used. Either one type of titanium-titanium dioxide sinter or a mixture of two or more types of titanium-titanium dioxide sinter can be used. Commercially available products of titanium-titanium dioxide sinter such as TILACK D (manufactured by Ako Kasei Co., Ltd.) and the like can also be used as the component (b).

The content of the above titanium-titanium dioxide sinter of the component (b) in the cosmetic pack preparation of the present invention is preferably 0.01-10%, and more preferably 0.1-5%. If less than 0.01%, the covering effect is insufficient to clearly identify the area coated with the cosmetic pack preparation and the film strength improving effect is small. If more than 10%, spreadability is insufficient.

Although the cosmetic pack preparation of the present invention can be prepared from the above components (a), (b), and water as essential components, various other components may be added to increase the action and effect of the cosmetic pack preparation.

Anionic surfactants (component (c)) can be mentioned as an example of such a component. The addition of the component (c) can synergistically increase the dispersion stability of the component (b).

There are no specific limitations to the anionic surfactant of the component (c) as long as the anionic surfactant is commonly used in cosmetic compositions. A fatty-acid soap anionic surfactant, alkyl ether carboxylate anionic surfactant, acylamino acid salt anionic surfactant, phosphate anionic surfactant, sulfonate anionic surfactant, and sulfate anionic surfactant can be given as examples.

Among these, phosphate anionic surfactants such as monolauryl sodium phosphate, diethanolamine monolauryl phosphate, triethanolamine monolauryl phosphate, potassium monolauryl phosphate, sodium monooleyl phosphate, sodium monopolyoxyethylene lauryl ether phosphate, sodium monopolyoxyethylene myristyl ether phosphate, sodium monopolyoxyethylene cetyl ether phosphate, sodium monopolyoxyethylene oleyl ether phosphate, sodium dipolyoxyethylene lauryl ether phosphate, sodium dipolyoxyethylene myristyl ether phosphate, sodium dipolyoxyethylene cetyl ether phosphate, and sodium dipolyoxyethylene oleyl ether phosphate; sulfonates anionic surfactants such as alkylbenzene sulfonate, olefin sulfonates, alkane sulfonates, alkyl sulfosuccinates, acyl isethionates, and acyl taurates; and sulfate anionic surfactants such as alkyl sulfates, alkenyl sulfates, and ethylene oxide or propylene oxide addition alkyl or alkenyl ether sulfates are preferable to ensure excellent dispersion stability. When a water-soluble ascorbic acid derivative or a water-soluble glycyrrhizic acid derivative (component (e)), which are mentioned later, is added, the use of dioctyl sodium sulfosuccinate as a component (c) is particularly preferable to promote dispersion stability of the component (b). Either one type or a mixture of two or more types of the above component (c) can be used.

Although not specifically limited, the content of the component (c) in the cosmetic pack preparation of the present invention is preferably 0.001-5%, and more preferably 0.01-1%, to ensure the effect of promoting the dispersion stability.

A water-soluble thickener may further be added to the cosmetic pack preparation of the present invention as component (d). The component (d) prevents dripping and the like of the cosmetic pack preparation, makes application of the cosmetic pack preparation to the skin easy, and remarkably improves the dispersion stability of the component (b).

Any water-soluble thickeners commonly used in cosmetic compositions can be used as the component (d) without specific limitations. A water-soluble polymer, a water swelling clay mineral, fine particles of silicic anhydride, and the like can be given as examples. Water swelling clay minerals such as bentonite, montmorillonite, hectorite, synthetic smectite, and water swelling fluorine mica and fine particles of amorphous silicic anhydride such as aluminum oxide-treated fine particles of amorphous silicic anhydride and sililated fine particles of amorphous silicic anhydride are particularly preferable.

As a specific component (d), water swelling clay minerals such as Kunipure G-4, SumectonSA-2 (manufactured by Kunimine Industries Co., Ltd.), BENTONE MA, EW, LT, RV (manufactured by Elementis Co.), and SUBMICA E (manufactured by Daito Chemical Industry, Co., Ltd.), and fine particles of amorphous silicic anhydride such as AEROSIL 200, 300, R972, R974, RX300 (manufactured by Nippon Aerosil Co., Ltd.) can be given. Either one type or a mixture of two or more types of the above component (d) can be appropriately selected and used.

Although not specifically limited, the content of the component (d) in the cosmetic pack preparation of the present invention is preferably 0.01-10%, and more preferably 0.1-5%, to ensure the effect of promoting the usability and dispersion stability.

It is also possible to add a water-soluble ascorbic acid derivative having a whitening effect and a water-soluble glycyrrhizic acid derivative exhibiting an anti-inflammatory effect as a component (e) to the cosmetic pack preparation of the present invention in a stable manner to the extent that the effect of the present invention is not impaired. Specifically, the combined use of the component (a) and component (b) makes it possible to add a water-soluble ascorbic acid derivative or a water-soluble glycyrrhizic acid derivative, both being electrolytes, as the component (e) without impairing the preservation stability of the cosmetic pack preparation.

Any water-soluble ascorbic acid derivative commonly used in whitening cosmetic compositions can be used as the component (e) without any specific limitations. As specific examples, ascorbic acid salts such as sodium L-ascorbate, potassium L-ascorbate, and magnesium L-ascorbate; ascorbyl phosphate such as sodium L-ascorbyl-2-phosphate, magnesium L-ascorbyl-2-phosphate, and calcium L-ascorbyl-2-phosphate; ascorbyl sulfate such as sodium L-ascorbyl-2-sulfate, magnesium L-ascorbyl-2-sulfate, and calcium L-ascorbyl-2-sulfate; and the like can be given. Also, any water-soluble glycyrrhizic acid derivative commonly used in anti-inflammatory cosmetic compositions can be used as the component (e) without any specific limitations. As specific examples, glycyrrhizic acid, glycyrrhetinic acid, ammonium glycyrrhizate, trisodium glycyrrhizate, dipotassium glycyrrhizate, and the like can be given. Either one type or a mixture of two or more types of the above component (e) can be appropriately selected and used.

Although not specifically limited, the content of the component (e) in the cosmetic pack preparation of the present invention is preferably 0.001-5%, and more preferably 0.01-3%.

In addition to the above components, a polyglycerol fatty acid ester can be added to the cosmetic pack preparation as a component (f) to synergistically increase the dispersion stability of the component (b).

Any polyglycerol fatty acid ester commonly used as a component of cosmetic compositions can be used as the component (f) without any specific limitations. As specific examples, decaglyceryl monolaurate, decaglyceryl monostearate, decaglyceryl monoisostearate, decaglyceryl monooleate, decaglyceryl distearate, decaglyceryl diisostearate, decaglyceryl dioleate, decaglyceryl tristearate, decaglyceryl triisostearate, decaglyceryl trioleate, decaglyceryl pentastearate, decaglyceryl pentaisostearate, decaglyceryl pentaoleate, hexacaglyceryl monolaurate, hexaglyceryl monostearate, hexaglyceryl monoisostearate, hexaglyceryl monooleate, and the like can be given. Either one type of polyglycerol fatty acid ester or a mixture of two or more types of polyglycerol fatty acid ester can be used.

Although not specifically limited, the content of the component (f) in the cosmetic pack preparation of the present invention is preferably 0.001-5%, and more preferably 0.01-3%, to ensure the effect of promoting the dispersion stability.

The cosmetic pack preparation of the present invention can be prepared by mixing the above essential components (a), (b), and water, and optional components (c) to (f) by a conventional method.

For example, a method of dissolving the component (a) in water by heating, adding to the resulting solution a homogeneous dispersion prepared by mixing the component (b), optionally together with a polyhydric alcohol and the components (c) and (f) using a three-way roll mill or the like, adding the components (d) and (e), if necessary, and homogeneously mixing the resulting mixture; a method of homogeneously mixing a portion of the component (a) with the component (b) and water using a three-way roll mill or the like to obtain a homogeneous dispersion and adding this dispersion to a solution prepared by dissolving the remaining portion of the component (a) and the components (c) to (f) in water with heating; and the like can be given.

In addition to the above-described components, other components commonly used in cosmetic preparations, for example, surfactants other than those mentioned above, aqueous components such as a polyhydric alcohol, antioxidants, UV absorbers, perfumes, antiseptic agents, coloring agents such as a pigment or dye other than those mentioned above, beauty ingredients, and the like can be added to the cosmetic pack preparation of the present invention to the extent that the effect of the present invention is not adversely affected.

### EXAMPLES

The present invention will be described in more detail by way of Examples which should not be construed as limiting the present invention.

### Examples 1-14 and Comparative Examples 1-6

Cosmetic Pack Preparation:

Cosmetic pack preparations were prepared from the components listed in the following Tables 1 and 2 using the following method of preparation. Evaluation items of the cosmetic pack preparations, i.e. (1) applicability, (2) identifiability of applied area, peeled area, and skin cleaning effect, (3) peelability, and (4) preservation stability, were evaluated according to the following evaluation method. The results are shown in Tables 1 and 2.

TABLE 1
%

| | | | Example | | | | | | | | | | | | | |
|---|---|---|---|---|---|---|---|---|---|---|---|---|---|---|---|---|
| | | | 1 | 2 | 3 | 4 | 5 | 6 | 7 | 8 | 9 | 10 | 11 | 12 | 13 | 14 |
| | Component | | | | | | | | | | | | | | | |
| 1 | Polyvinyl alcohol (*1) | | — | 0.34 | 10.0 | 5.0 | 20.0 | 5.0 | 5.0 | 1.0 | 7.5 | 0.2 | 0.2 | 0.2 | 5.0 | 5.0 |
| 2 | Polyvinyl alcohol (*2) | | 5.0 | 6.66 | 5.0 | 5.0 | — | 5.0 | 5.0 | 10.0 | 5.0 | 6.0 | 6.0 | 6.0 | 5.0 | 5.0 |
| 3 | Titanium-titanium dioxide | | 2.0 | 0.1 | 5.0 | 2.0 | 2.0 | 0.01 | 10.0 | 2.0 | 2.0 | 2.0 | 2.0 | 2.0 | 9.0 | 9.0 |
| | sinter (*3) | | | | | | | | | | | | | | | |
| 4 | Black iron oxide | | — | — | — | — | — | — | — | — | — | — | — | — | — | — |
| 5 | Black iron oxide-treated mica | | — | — | — | — | — | — | — | — | — | — | — | — | — | — |
| 6 | Decaglyceryl monooleate | | 0.5 | 0.5 | 0.5 | 0.5 | 0.5 | 0.5 | 0.5 | 0.5 | 0.5 | 0.5 | 0.5 | 0.5 | 0.5 | 0.5 |
| 7 | Sodium dipolyoxyethylene | | — | 0.001 | 1.0 | 0.5 | — | — | — | 0.5 | 0.5 | — | — | 0.01 | 0.5 | 5.0 |
| | lauryl ether phosphate | | | | | | | | | | | | | | | |
| 8 | Ethanol | | 10.0 | 10.0 | 10.0 | 10.0 | 10.0 | 10.0 | 10.0 | 10.0 | 10.0 | 10.0 | 10.0 | 10.0 | 10.0 | 10.0 |
| 9 | 1,3-Butylene glycol | | 5.0 | 5.0 | 5.0 | 5.0 | 5.0 | 5.0 | 5.0 | 5.0 | 5.0 | 5.0 | 5.0 | 5.0 | 5.0 | 5.0 |
| 10 | Fine particles of amorphous | | — | 0.01 | 5.0 | 1.0 | — | — | — | 1.0 | 1.0 | — | 0.1 | 0.1 | 10.0 | 1.0 |
| | silicic anhydride (*4) | | | | | | | | | | | | | | | |
| 11 | Purified water | | (**1) | (**1) | (**1) | (**1) | (**1) | (**1) | (**1) | (**1) | (**1) | (**1) | (**1) | (**1) | (**1) | (**1) |
| 12 | Antiseptic agent | | (**2) | (**2) | (**2) | (**2) | (**2) | (**2) | (**2) | (**2) | (**2) | (**2) | (**2) | (**2) | (**2) | (**2) |
| 13 | Perfume | | (**2) | (**2) | (**2) | (**2) | (**2) | (**2) | (**2) | (**2) | (**2) | (**2) | (**2) | (**2) | (**2) | (**2) |
| Evaluation items | | | Evaluation Result | | | | | | | | | | | | | |
| 1 | Applicability | | ◯ | ◯ | ● | ● | ◯ | ◯ | ◯ | ● | ● | ◯ | ● | ● | ◯ | ◯ |
| 2 | Identifiability of applied area, | | ● | ● | ● | ● | ● | ◯ | ● | ● | ● | ● | ● | ● | ● | ● |
| | peeled area, and skin cleaning | | | | | | | | | | | | | | | |
| | effect | | | | | | | | | | | | | | | |
| 3 | Peelability | | ◯ | ● | ● | ● | ● | ● | ● | ● | ● | ◯ | ◯ | ◯ | ● | ● |
| 4 | Preservation | 40° C. | ◯ | ● | ● | ● | ● | ● | ● | ● | ● | ● | ● | ● | ● | ● |
| | stability | 5° C. | ● | ● | ● | ● | ◯ | ● | ◯ | ● | ● | ● | ● | ● | ◯ | ◯ |

(*1) PVA-205C; manufactured by Kuraray Co., Ltd. (Viscosity (4%, 20° C.): 5.0-6.0 mPa · s)
(*2) PVA-224C; manufactured by Kuraray Co., Ltd. (Viscosity (4%, 20° C.): 42.0-50.0 mPa · s)
(*3) TILACK D; manufactured by Ako Kasei Co., Ltd.
(*4) AEROSIL 200; manufactured by Nippon Aerosil Co., Ltd.
(**1) Balance
(**2) Appropriate amount

TABLE 2

| | | | Comparative Example | | | | | |
|---|---|---|---|---|---|---|---|---|
| | | | 1 | 2 | 3 | 4 | 5 | 6 |
| | Component | | | | | | | |
| 1 | Polyvinyl alcohol (*1) | | — | 25.0 | 5.0 | 5.0 | 5.0 | 5.0 |
| 2 | Polyvinyl alcohol (*2) | | 3.0 | — | 5.0 | 5.0 | 5.0 | 5.0 |
| 3 | Titanium-titanium | | 2.0 | 2.0 | 0.001 | 15.0 | — | — |
| | dioxide sinter (*3) | | | | | | | |
| 4 | Black iron oxide | | — | — | — | — | 2.0 | — |
| 5 | Black iron oxide- | | — | — | — | — | — | 2.0 |
| | treated mica | | | | | | | |
| 6 | Decaglyceryl | | 0.5 | 0.5 | 0.5 | 0.5 | 0.5 | 0.5 |
| | monooleate | | | | | | | |
| 7 | Sodium | | — | — | — | — | — | — |
| | dipolyoxyethylene | | | | | | | |
| | lauryl ether phosphate | | | | | | | |
| 8 | Ethanol | | 10.0 | 10.0 | 10.0 | 10.0 | 10.0 | 10.0 |
| 9 | 1,3-Butylene glycol | | 5.0 | 5.0 | 5.0 | 5.0 | 5.0 | 5.0 |
| 10 | Fine particles of | | — | — | — | — | — | — |
| | amorphous silicic | | | | | | | |
| | anhydride (*4) | | | | | | | |
| 11 | Purified water | | (**1) | (**1) | (**1) | (**1) | (**1) | (**1) |
| 12 | Antiseptic agent | | (**2) | (**2) | (**2) | (**2) | (**2) | (**2) |
| 13 | Perfume | | (**2) | (**2) | (**2) | (**2) | (**2) | (**2) |
| Evaluation items | | | Evaluation Result | | | | | |
| 1 | Applicability | | ◯ | X | ● | X | Δ | Δ |
| 2 | Identifiability of | | ● | ● | X | Δ | ● | ● |
| | applied area, peeled | | | | | | | |
| | area, and skin clearing | | | | | | | |
| | effect | | | | | | | |
| 3 | Peelability | | X | Δ | Δ | Δ | ◯ | ◯ |
| 4 | Preservation | 40° C. | ● | ● | ● | ● | X | X |
| | stability | 5° C. | ● | X | ● | X | Δ | Δ |

(*1) PVA-205C; manufactured by Kuraray Co., Ltd. (Viscosity (4%, 20° C.): 5.0-6.0 mPa · s)
(*2) PVA-224C; manufactured by Kuraray Co., Ltd. (Viscosity (4%, 20° C.): 42.0-50.0 mPa · s)
(*3) TILACK D; manufactured by Ako Kasei Co., Ltd.
(*4) AEROSIL 200; manufactured by Nippon Aerosil Co., Ltd.
(**1) Balance
(**2) Appropriate amount

(Method of Preparation)
A: Components 3,4,5,6,7, and 9 are dispersed homogeneously using a three-way roll mill.
B: Components 1, 2, 8, 10, 11, and 12 are dissolved by heating at 80° C.
C: After cooling B to room temperature, A and component 13 are added and the mixture is mixed homogeneously.
D: A container is filled with C to obtain a cosmetic pack preparation.
   (Method of Evaluation)
   Evaluation of Evaluation Items 1-3:

20 women who had used cosmetics more than 10 years were selected as panelists. After using each cosmetic pack preparation for 14 days, these panelists evaluated items of the preparations, i.e. (1) applicability, (2) identifiability of applied area, peeled area, and skin cleaning effect, and (3) peelability according to the following five-grade absolute criterion. The average of scores for each obtained sample was used for judgment according to the following four-grade judgment standard.

(i) Five-Grade Absolute Criterion

| (Score) | (Evaluation) |
|---|---|
| 5 | Very good |
| 4 | Good |
| 3 | Fair |
| 2 | Bad |
| 1 | Extremely bad |

(ii) Four-Grade Judgment Standard

| (Score average) | (Judgment) |
|---|---|
| 4.5 or more | ● |
| 3.5 or more, but less than 4.5 | ◯ |
| 2.5 or more, but less than 3.5 | Δ |
| Less than 2.5 | X |

Evaluation of Evaluation Item 4:

Changes in the conditions of fine-particle dispersion and gelatinization in the cosmetic pack preparations preserved for one month at 40° C. after preparation and the cosmetic preparations preserved for one month at 5° C. after preparation were observed with the naked eye to judge preservation stability according to the following four-stage judgment standard.

(iii) Four-Grade Judgment Standard

| (Evaluation) | (Judgment) |
|---|---|
| No change: | ● |
| Slight change: | ◯ |
| Some degree of change: | Δ |
| Considerable degree of change: | X |

As is clear from the results shown in the above Tables 1 and 2, the cosmetic pack preparations of Examples 1-14 in the present invention were superior to the cosmetic pack preparations of Comparative Examples 1-6 in terms of clear identifiability of applied area, peeled area, and skin cleaning effect, favorable usability such as applicability and peelability, and excellent preservation stability with passage of time.

### Example 15

Peel-Off Type Cosmetic Pack Preparation:

A peel-off type cosmetic pack preparation was prepared using the following formulation and the following preparation method.

<Formulation>

| (Component) | (%) |
|---|---|
| 1. Polyvinyl alcohol (*5) | 10.0 |
| 2. Polyvinyl alcohol (*6) | 7.0 |
| 3. Bentonite | 1.0 |
| 4. Ethanol | 10.0 |
| 5. 1,3-Butylene glycol | 5.0 |
| 6. Talc | 2.0 |
| 7. Titanium-titanium dioxide sinter (*3) | 1.0 |
| 8. Sorbitan sesquioleate | 0.5 |
| 9. Polyglyceryl monolaurate | 0.5 |
| 10. Dioctyl sodium sulfosuccinate | 0.5 |
| 11. Sodium L-ascorbyl-2-phosphate | 1.0 |
| 12. Purified water | Balance |
| 13. Methyl parahydroxybenzoate | 0.2 |

(*3): Same as above
(*5): Gosenol EG-05 (manufactured by Nippon Synthetic Chemical Industry Co., Ltd.) (Viscosity (4%, 20° C.): 4.8-5.8 mPa · s)
(*6): Gosenol EG-40 (manufactured by Nippon Synthetic Chemical Industry Co., Ltd.) (Viscosity (4%, 20° C.): 40.0-46.0 mPa · s)

Manufacturing Method
A: Components 5, 6, 7, 8, 9, and 10 are dispersed homogeneously using a three-way roll mill.
B: Components 1, 2, 3, 4, 11, 12, and 13 are dissolved by heating at 80 ° C.
C: After cooling B to room temperature, A is added and the mixture is mixed homogeneously.
D: A container is filled with C to obtain a peel-off type cosmetic pack preparation.

The peel-off type cosmetic pack preparation of Example 15 was confirmed to provide clear identifiability of applied area, peeled area, and skin cleaning effect and favorable usability such as applicability and peelability and to possess excellent preservation stability with passage of time as well as an excellent whitening effect.

### Example 16

Peel-Off Type Cosmetic Pack Preparation:

A peel-off type cosmetic pack preparation was prepared using the following formulation and the following preparation method.

<Formulation>

| (Component) | (%) |
|---|---|
| 1. Polyvinyl alcohol (*1) | 2.0 |
| 2. Polyvinyl alcohol (*2) | 12.0 |
| 3. Sililated fine particles of amorphous silicic anhydride (*7) | 2.0 |
| 4. Ethanol | 10.0 |
| 5. 1,3-Butylene glycol | 1.0 |
| 6. Talc | 2.0 |
| 7. Titanium-titanium dioxide sinter (*3) | 1.0 |
| 8. Silicic anhydride | 3.0 |
| 9. Dioctyl sodium sulfosuccinate | 0.1 |
| 10. Decaglyceryl monoisostearate | 0.5 |
| 11. Dipotassium glycyrrhizate | 0.1 |
| 12. Purified water | Balance |
| 13. Methyl parahydroxybenzoate | 0.2 |

(*1), (*2) and (*3): Same as above
(*7): AEROSIL R972 (manufactured by Nippon Aerosil Co., Ltd.)

Manufacturing Method
A: Components 5, 6, 7, and 12 are dissolved by heating, a component 1 is added, and the mixture was dispersed homogeneously using a three-way roll mill.
B: Components 2-4 and 8-13 were heated and dissolved at 80° C.
C: B was cooled to room temperature, A was added, and the mixture was homogeneously mixed.
D: A container is filled with C to obtain a cosmetic pack preparation.

The peel-off type cosmetic pack preparation of Example 16 was confirmed to provide clear identifiability of applied area, peeled area, and skin cleaning effect and favorable usability such as applicability and peelability and to possess excellent preservation stability with passage of time as well as an excellent anti-inflammatory effect.

### INDUSTRIAL APPLICABILITY

A film forming-type cosmetic pack preparation of the present invention can provide distinct identifiable areas which the cosmetic pack preparation was applied to and was peeled from and the skin cleaning effect, imparts a favorable sensation upon use such as ease of application and peeling, and excels in preservation stability with passage of time.

Due to the above-mentioned properties, the cosmetic pack preparation in the present invention can be widely used as a pack agent to supply moisture to the skin and suppress vaporization of subcutaneous moisture and to make skin flexible, a pack agent to cause a medicinal component to be absorbed in the skin, and the like.

What is claimed is:

1. A cosmetic pack preparation comprising (a) 5-20 wt % of polyvinyl alcohol and (b) 0.01-10 wt % of a titanium-titanium dioxide sinter.

2. The cosmetic pack preparation according to claim 1, further comprising an anionic surfactant as a component (c).

3. The cosmetic pack preparation according to claim 2, wherein the anionic surfactant of the component (c) is one or more anionic surfactants selected from the group consisting of a phosphate-based anionic surfactant, a sulfonate-based anionic surfactant, and a sulfate-based anionic surfactant.

4. The cosmetic pack preparation according to any one of claims 1-3, further comprising a water-soluble thickener as a component (d).

5. The cosmetic pack preparation according to claim 4, wherein the compound (d) is a water swelling clay mineral and/or fine particles of amorphous silicic anhydride.

6. The cosmetic pack preparation according to any one of claims 1-5, wherein the polyvinyl alcohol of the component (a) is a mixture of (A) a polyvinyl alcohol of which the aqueous solution with a concentration of 4 wt % has a viscosity at 20° C. of 1 mPa.s or more, but less than 30 mPa.s and (B) a polyvinyl alcohol of which the aqueous solution with a concentration of 4 wt % has a viscosity at 20° C. of 30 mPa.s or more, but less than 70 mPa.s.

7. The cosmetic pack preparation according to claim 6, wherein the weight ratio of the polyvinyl alcohol (A) to the polyvinyl alcohol (B) in the component (a) is 0.05-2.

8. The cosmetic pack preparation according to any one of claims 1-7, further comprising a water-soluble ascorbic acid derivative and/or a water-soluble glycyrrhizic acid derivative as a component (e).

9. The cosmetic pack preparation according to any one of claims 1-8, further comprising a polyglycerol fatty acid ester as a component (f).

10. The cosmetic pack preparation according to any one of claims 1-9, wherein the anionic surfactant of the component (c) is dioctyl sodium sulfosuccinate.
